(19) United States
(12) Patent Application Publication
(10) Pub. No.: US 2013/0261706 A1
(43) Pub. Date: Oct. 3, 2013

(54) SYSTEMS AND METHODS FOR APPLYING RAPID SEQUENTIAL ELECTRODE STIMULATION

(71) Applicants: Emily A. MIRRO, San Francisco, CA (US); Stephen T. Archer, Sunnyvale, CA (US)

(72) Inventors: Emily A. MIRRO, San Francisco, CA (US); Stephen T. Archer, Sunnyvale, CA (US)

(73) Assignee: NeuroPace, Inc., Mountain View, CA (US)

(21) Appl. No.: 13/444,683

(22) Filed: Apr. 11, 2012

Related U.S. Application Data
(60) Provisional application No. 61/618,565, filed on Mar. 30, 2012.

(51) Int. Cl. A61N 1/36

(52) U.S. Cl. 607/74

(57) ABSTRACT

Described herein are methods and systems for delivering a burst of stimulation pulses or pulse segments sequentially to a plurality of stimulation pathways. The stimulation pulses may be generated by a stimulation device, which may comprise an implantable neurostimulator. The stimulation pathways may comprise one or more electrodes electrically connected to the stimulation device. In some variations, the stimulation pathway may comprise a monopolar stimulation pathway and/or a bipolar stimulation pathway.

### CROSS-REFERENCE TO RELATED APPLICATIONS

This application claims priority to U.S. Provisional Application Ser. No. 61/618,565, filed on Mar. 30, 2012, which is hereby incorporated by reference in its entirety.

### FIELD

The devices and methods described here are related to systems and methods for providing stimulation to tissue.

### BACKGROUND

Epileptic seizures are associated with excessive or abnormally synchronous neuronal activity. Physicians have been able to treat epilepsy by surgery to resect one or more brain portions or by medication. Brain surgery is irreversible, and may be ineffective or associated with neural morbidity in a sizable percentage of cases. In many instances, medication may be ineffective in controlling seizures, or patients may suffer from debilitating side effects. A more promising method of treating patients having epileptic seizures is by electrical stimulation of the brain.

Devices and methods for delivering electrical stimulation through electrodes have been used to treat epilepsy, as well as a number of other conditions, including chronic pain, cardiac arrhythmias, and the like. It may be desirable to provide one or more improvements to stimulation devices and methods.

### BRIEF SUMMARY

Described here are methods of rapid sequential electrode stimulation (“RSES”) and systems for performing RSES. In some variations, the methods may comprise delivering a plurality of stimulation pulses to a patient by delivering the pulses or segments thereof sequentially to a plurality of stimulation pathways. The stimulation pulses may be generated and delivered by a stimulation system. In some variations, the stimulation system may comprise a stimulation device and one or more leads containing one or more electrodes. In some variations, the stimulation device may comprise a neurostimulator. In other variations, the stimulation device may comprise a spinal cord stimulator, a pacemaker, an implantable cardioverter defibrillator, or the like. For each pulse or segment of a pulse, the stimulation device may be configured to select an individual stimulation pathway, such that the pulse or segment of the pulse may be delivered to a single stimulation pathway at any given moment in time, such that stimulation is applied to one stimulation pathway at a time. The plurality of stimulation pulses (or segments of the plurality of stimulation pulses) may be sequentially delivered to any number of stimulation pathways (e.g., two, three, four, or five or more stimulation pathways). In some variations, the stimulation pulses may be delivered to a first stimulation pathway and a second stimulation pathway. In these variations, a first pulse may be delivered to the first stimulation pathway and a second pulse may be subsequently delivered to a second stimulation pathway. The sequential delivery of stimulation pulses to the first and second stimulation pathways may be repeated until each of the stimulation pulses have been delivered to the tissue. In other variations, the stimulation pulses may be sequentially delivered to four stimulation pathways. In these variations, a first pulse may be delivered to tissue via a first stimulation pathway, a second pulse may subsequently be delivered to tissue via a second stimulation pathway, a third pulse may be subsequently delivered to tissue via a third stimulation pathway, and a fourth pulse may be subsequently delivered to tissue via a fourth stimulation pathway. The sequential delivery of stimulation pulses to the first, second, third, and fourth stimulation pathways may be repeated until each of the stimulation pulses have been delivered to tissue. In other variations, the stimulation pulses are divided into a plurality of pulse segments, such that the pulse segments are sequentially delivered to the plurality of stimulation pathways. For example, the positive phase of a biphasic pulse may be divided into first and second pulse segments, and a negative phase of the biphasic pulse may be divided into first and second pulse segments. In some of these variations, delivery of the biphasic pulse may comprise introducing the first segment of the positive phase to a first stimulation pathway, introducing the second segment of the positive phases to a second stimulation pathway, introducing the first segment of the negative phase to the first stimulation pathway, and introducing the second segment of the negative phase to the second stimulation pathway.

The stimulation pathways described here may be defined by one or more electrodes. In some variations, a stimulation pathway may be a monopolar stimulation pathway, in which the stimulation pathway comprises a first electrode electrically connected to a stimulation device via a lead and a second reference electrode. In some of these variations, the reference electrode may be one or more conductive portions of a stimulation device (e.g., a conductive portion of a housing of the stimulation device). In some variations, a stimulation pathway may be a bipolar stimulation pathway, which may comprise a first electrode and a second electrode electrically connected to a stimulation device via one or more leads. In some of these variations, the first and second electrodes are located on the same lead. In others of these variations, the first and second electrodes are located on different leads. A plurality of stimulation pathways may comprise any combination of monopolar and/or bipolar stimulation pathways.

Also described here are systems for delivering rapid sequential electrode stimulation. In some variations, the systems may comprise a stimulation device configured to generate a plurality of stimulation pulses, and a plurality of electrodes which may define a plurality of stimulation pathways. The stimulation systems may be programmed to deliver the stimulation pulses or segments thereof sequentially to each of the plurality of stimulation pathways using one or more of the methods as described hereinthroughout. In some variations, the stimulation device comprises an implantable neurostimulator. The neurostimulator may comprise any combination of subsystems, including a stimulation subsystem, detection subsystem, CPU, memory subsystem, and/or communication subsystem, as will be described in more detail below.

### BRIEF DESCRIPTION OF THE DRAWINGS

FIGS. 1A and 1B depict an illustrative variation of a pulse and a burst of pulses, respectively, that may be delivered using the systems and methods described here.

FIG. 2A depicts an illustrative variation of the stimulation systems described herein. FIG. 2B depicts a method by which the stimulation system of FIG. 2A may deliver stimulation to tissue.

FIG. 3A depicts an illustrative variation of the stimulation systems described herein. FIG. 3B depicts a method by which the stimulation system of FIG. 3A may deliver stimulation to tissue.

FIG. 4A depicts an illustrative variation of the stimulation systems described herein. FIG. 4B depicts a method by which the stimulation system of FIG. 4A may deliver stimulation to tissue.

FIG. 5A depicts an illustrative variation of the stimulation systems described herein. FIG. 5B depicts a method by which the stimulation system of FIG. 5A may deliver stimulation to tissue.

FIG. 6 shows a block diagram of a variation of a neurostimulator that may provide neurostimulation to a patient.

FIG. 7 depicts a perspective view of an implantable neurostimulation system suitable for use with the devices and methods described here.

FIGS. 8, 9A, 9B, 10A, and 10B depict methods by which a stimulation system may deliver stimulation to tissue.

### DETAILED DESCRIPTION OF THE INVENTION

Described here are methods of rapid sequential electrode stimulation (“RSES”) and systems for performing RSES. Generally, the RSES methods described here comprise delivering a plurality of stimulation pulses to tissue by delivering the pulses or segments of the pulses sequentially to each of a plurality of stimulation pathways, in which stimulation is provided to a single stimulation at each moment in time. Generally, one or more stimulation systems may be configured to generate and apply the stimulation pulses to the plurality of the stimulation pathways according to one or more of the RSES methods described here. The stimulation systems may comprise one or more stimulation devices, which may be one or more neurostimulators, spinal cord stimulators, pacemakers, implantable cardioverter defibrillators or the like, as will be described in more detail below. The stimulation pathways may comprise one or more electrodes, some or all of which may be connected to the stimulation device via one or more leads. Stimulation using the RSES methods described here may reduce the power consumption of a stimulation device and/or may help provide more uniform stimulation of tissue, as will be described in more detail below.

As mentioned above, the RSES methods may comprise delivering one or more stimulation pulses to tissue. Specifically, the stimulation devices described here may be configured to generate (e.g., via a stimulation subsystem) a set of stimulation pulses (also called a “burst”). A burst may comprise any suitable number of pulses (e.g., 1, 2, 50, 100, 200 or more pulses), and may be generated at any suitable frequency or frequencies (e.g., from 0 Hz (i.e., effectively DC stimulation) through about 0.5 Hz and beyond, including frequencies in the megahertz range and higher), as will be described in more detail below. The stimulation systems may be configured to generate and deliver RSES stimulation in any suitable manner. In some variations, stimulation may be delivered in a non-responsive or open-loop manner, wherein the stimulation is delivered on a scheduled basis. In these variations, stimulation may be delivered continuously or on a periodic basis. In some variations, one or more parameters of the stimulation may be varied based on time of day, or circadian rhythms (e.g., in some patients it may be advantageous to alter stimulation patterns before or during normal sleep times to avoid disrupting sleep patterns). Additionally or alternatively, stimulation may be delivered in a responsive or closed-loop manner, in which stimulation is delivered in response to a condition determined by the stimulation system or another system distinct from the stimulation system. For example, the stimulation system may be configured to measure one or more physiological parameters (e.g., one or more electrophysiological signals, temperature, blood pressure, or the like), and may deliver stimulation when the stimulation system detects one or more pre-determined criteria (e.g., one or more threshold values and/or one or more patterns) in the measured parameter.

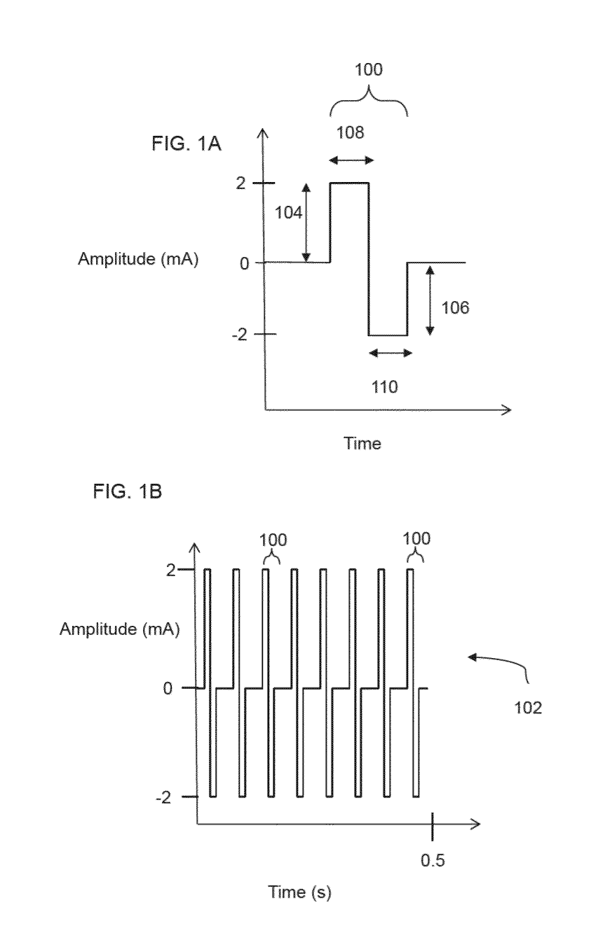

The stimulation systems may be configured to produce any suitable pulses or combination of pulses. A stimulation pulse may be monophasic or biphasic, and in some instances may be charge-balanced. The pulse may have any suitable pulse morphology, and each phase of the pulse may be square, triangular, trapezoidal, haversine, or another shape. FIG. 1A shows one variation of a biphasic square pulse (100) having a first-phase amplitude (104), a second-phase amplitude (106), a first-phase pulse width (108), and a second-phase pulse width (110). In instances where the stimulation is delivered to neural tissue in a human patient's brain to treat epilepsy, typical pulse amplitude and pulse width ranges are 0.5 mA-12 mA and 40 μs-500 μs respectfully. For the purposes of illustration and for discussion of the stimulation system variations described below, the first-phase amplitude (104) and the second-phase amplitude (106) are shown in FIG. 1A as being +2 mA and −2 mA, respectively, and the first-phase pulse width (108) and the second-phase pulse width (110) are each shown there as 160 μs. While the first and second phases of the pulse (100) are shown in FIG. 1A are shown as having the same amplitude, shape, and pulse width, it should be appreciated that different phases may have different amplitudes, shapes, and/or pulse widths. Additionally, while the pulse (100) shown in FIG. 1A having +/−2 mA first and second phase amplitudes and the same pulse width for each phase is charge-balanced, the pulse (100) need not be.

FIG. 1B shows a time graph of a burst (102) of pulses (100) which may be delivered by one or more of the stimulators described here. The burst (102) may comprise any suitable number of pulses (100), and the pulses (100) may be delivered at any suitable frequency. In instances where stimulation is delivered to the neural tissue in a human patient's brain to treat epilepsy, typical frequencies may range from 1 Hz to 333 Hz. For the purposes of illustration and for discussion of the stimulation system variations described below, the pulses (100) of the burst (102) are shown in FIG. 1B as being delivered at a frequency of 16 Hz for a duration of 0.5 second. Thus, for the 0.5 second burst shown in FIG. 1B, 8 pulses (100) may be delivered. While the inter-pulse interval between each pulse (100) of the burst (102) is shown in FIG. 1B as being constant, it should be appreciated that the inter-pulse interval within the burst (102) may vary over the course of delivery of the burst (102). In some variations, the frequency of pulses may vary from burst to burst and/or the interval between pulses with a given burst may be varied. Indeed, any of the parameters characterizing a pulse within a burst or the bursts themselves may be varied, for example, to be the same or different from pulse to pulse or from burst to burst. It should be appreciated that any or all of the characteristics of a burst and its individual pulses may be pre-programmed. In some variations, one or more characteristics of one or more pulses may be determined at least partially based on one or more signals or other parameters measured by the stimulating device.

The RSES methods and stimulation systems may comprise delivering one or more bursts of stimulation pulses to a plurality of stimulation pathways. In some variations, individual pulses may be divided such that segments of each pulse may be delivered to the plurality of stimulation pathways. A stimulation pathway generally comprises one or more electrodes, and may comprise an anode and a cathode such that current may pass through tissue between the anode and the cathode. In some variations, a given electrode may act as an anode or a cathode for multiple stimulation pathways, as will be described in more detail below. Some or all of the electrodes in a stimulation pathway may be electrically connected to a stimulation device via one or more leads. The plurality of stimulation pathways may comprise one or more monopolar stimulation pathways and/or one or more bipolar stimulation pathways, each of which will be described in more detail below. When the stimulation systems deliver a burst of stimulation pulses to tissue, the stimulation systems may be configured to select which pathway receives each pulse or segment thereof, as will be described in more detail below.

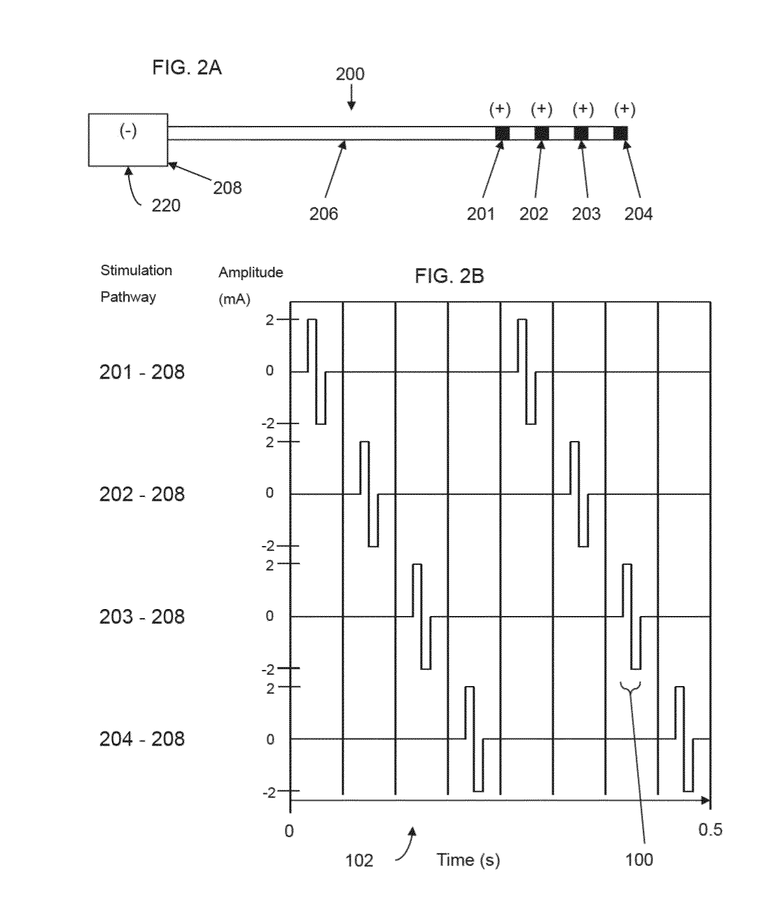

In variations where a stimulation pathway comprises a monopolar stimulation pathway, the stimulation pathway may comprise two electrodes. In these variations, a first electrode may be attached to a stimulation device via one or more leads, and the second electrode may be a reference electrode. The reference electrode may be any suitable electrode, such as, for example, a conductive portion of a housing of the stimulation device. The first electrode may be an anode and the reference electrode may be a cathode, or vice versa, such that during stimulation current may flow between the first electrode and the second electrode. FIGS. 2A and 2B illustrate one variation of a stimulation system (200) in which the stimulation system (200) comprises a plurality of monopolar stimulation pathways. As shown in FIG. 2A, the stimulation system (200) may comprise a stimulation device (220), and four electrodes (201)-(204) which may be connected to stimulation device (220) via a lead (206). As shown there, stimulation device (220) may further comprise a reference electrode (208), which in some variations may comprise a conductive portion of a housing (not shown) of the stimulation device (220). The stimulation device (220) may be any suitable stimulation device as described hereinthroughout.

Each of the electrodes (201)-(204) may act as a monopolar stimulation pathway with the reference electrode (208). For example, the stimulation device (220) may be configured to deliver one or more pulses or segments thereof to the electrode (201) and the reference electrode (208) as a first stimulation pathway, to the electrode (202) and the reference electrode (208) as a second stimulation pathway, to the electrode (203) and the reference electrode (208) as a third stimulation pathway, and to the electrode (204) and the reference electrode (208) as a fourth stimulation pathway. As shown in FIG. 2A, each of the electrodes (201)-(204) may be an anode (as indicated by a (+) sign) and the reference electrode (208) may be a cathode (as indicated by a (−) sign) in each of the four stimulation pathways. It should be appreciated, however, that one or more of the electrodes (201)-(204) may be a cathode for one or more of the stimulation pathways, with the reference electrode (208) being an anode for these stimulation pathways. The designation of an anode as a (+) sign and a cathode as a (−) sign are merely for convenience to provide an indication of the direction of the current flow through a patient between two electrodes. It should be appreciated that the convention of an anode being associated with a (+) sign and a cathode being associated with a (−) may be reversed in some circumstances.

The stimulation device (220) may be programmed and configured to sequentially deliver a stimulation signal to any of these stimulation pathways using a RSES method. FIG. 2B shows a time graph of one manner in which a RSES method may be used to deliver the burst (102) of pulses (100) of FIGS. 1A and 1B to tissue. In this method, the stimulation device (220) may be programmed and configured to deliver the pulses (100) sequentially to each of the four pathways in any suitable order, such that each pulse is only delivered to a single stimulation pathway. For example, in the variation shown in FIG. 2B, the stimulation device (220) may be configured to select a first stimulation pathway (e.g., the stimulation pathway (201)-(208)) and deliver a first pulse thereto. Following delivery of the first pulse, the stimulation device (220) may select a second stimulation pathway (e.g., the stimulation pathway (202)-(208)) and deliver a second pulse thereto. The stimulation device (220) may then select and deliver a third pulse to a third stimulation pathway (e.g., the stimulation pathway (203)-(208)), and then may select and deliver a fourth pulse to a fourth stimulation pathway (e.g., the stimulation pathway (204)-(208)). This pattern may be repeated one or more times until each pulse of the burst has been delivered to tissue. For example, when the stimulation system (200) is used to deliver the variation of the burst (102) described above with respect to FIG. 1B, the stimulation device (220) may repeat the pattern a second time, such that two pulses are delivered to each of the first, second, third, and fourth stimulation pathways over the 0.5 second burst.

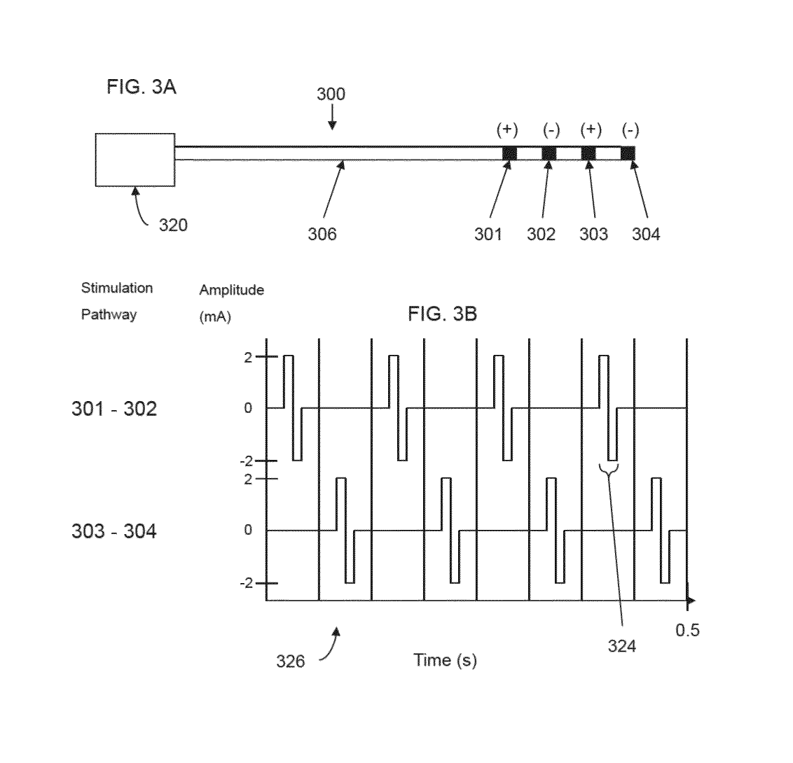

During sequential application of the pulses of a burst to a plurality of stimulation pathways, the pulses may be sequentially delivered to the stimulation pathways in any order as may be desired. For example, in some variations of the stimulation system (200) shown above in FIG. 2A, stimulation pulses may be applied to the four monopolar stimulation pathways in a left-to-right or right-to-left pattern across the electrodes (201)-(204). For example, in some variations, the first pathway to which a pulse (100) of the burst (102) described above with regard to FIG. 1B is applied may be stimulation pathway (201)-(208), the pathway between the reference electrode (208) and the electrode that is the least distal of the four electrodes from the stimulation device (220) (i.e., electrode (201)). The next pulse (100) of the burst (102) is applied to stimulation pathway (202)-(208), which is the pathway between the between the reference electrode (208) and the electrode that is the second least distal of the four electrodes from the stimulation device (220) (i.e., electrode (202)), the next pulse (100) of the burst (102) is applied to stimulation pathway (203)-(208), which is the pathway between the between the reference electrode (208) and the electrode that is the third least distal of the four electrodes from the stimulation device (220) (i.e., electrode (203)), the next pulse (100) of the burst (102) is applied to stimulation pathway (204)-(208), which is the pathway between the between the reference electrode (208) and the electrode that is the most distal of the four electrodes from the stimulation device (220) (i.e., electrode (204)), and then the sequence picks up with the pathway (201)-(208) and proceeds again through the four stimulation pathways until all of the pulses (100) in the burst (102) have been delivered. In other variations, the pulses may be applied in the reverse order or out of order, depending on how it is desired to stimulate the tissue that lies between the electrodes in each pathway. In some of these variations the pulses may be delivered in order in bipolar fashion from the distal-most electrode to the proximal-most electrode or from the proximal-most electrode to the distal-most electrode in a bipolar fashion. The latter variation is shown schematically in FIGS. 3A and 3B, where one pulse (324) of a burst (326) is delivered through a pathway comprising the least distal electrode (301) (+) and second least distal electrode the (302) (−) relative to a stimulation device (320), a next pulse of the burst (326) is delivered through a pathway comprising the third least distal electrode (303) and the most distal electrode (304), and then the sequence returns to and delivers the next pulse to pathway (301)-(302), and the next pulse to (303)-(304) and so on, until all of the pulses (324) in the burst (326) (in the example shown, eight pulses in total over the half second burst duration) are delivered.

While stimulation system (200) described with reference to FIG. 2A repeatedly uses four monopolar pathways to deliver the pulses (100) of a burst (102) and the stimulation system described with reference to FIG. 3A repeatedly uses two bipolar pathways, it should be appreciated that the stimulation systems described here may comprise any suitable number of stimulation pathways, such as, for example, two, three, four, five, six, seven, eight, or nine or more stimulation pathways and any combination of pathway configurations (e.g., monopolar (also referred to as “unipolar”), bipolar, or the like). For a given delivery of a burst, the stimulation system may be programmed to select the number of stimulation pathways to which individual pulses of the burst will be delivered, the electrodes that make up each stimulation pathway, and the order of pulse delivery to each of the electrodes. Any or all of these parameters may change between subsequent bursts, depending on the programming of the stimulation system. For example, in the variation of stimulation system (200) described above with respect to FIG. 2A, the stimulation device (220) may be configured to deliver pulses to different sets of stimulation pathways during different bursts. More specifically, the system may be programmed to define four monopolar pathways where each pathway is defined between one electrode of the four electrodes (201)-(204) on a distal end of an implanted brain lead as an anode, and a reference electrode (208) (e.g., the housing of the stimulation device (220)) as the cathode, or vice versa. The stimulation system further may be programmed with a first stimulation delivery sequence for a first burst comprised of a plurality of pulses which uses only three of the four pathways (e.g., stimulation pathways (201)-(208), (202)-(208), and (203)-(208)). Each pulse of the first burst may be applied to one of the three stimulation pathways (201)-(208), (202)-(208), and (203)-(208) according to the first stimulation delivery sequence. The stimulation system may further be programmed with a second stimulation delivery sequence for a second burst comprised of a plurality of pulses which may use a different subset of the four pathways (e.g., stimulation pathways (202)-(208), (203)-(208), and (204)-(208)), and each pulse of the second burst may be applied to one of the stimulation pathways (202)-(208), (203)-(208), and (204)-(208) according to the second stimulation delivery sequence. The stimulation system may be programmed with any number of stimulation delivery sequences.

Additionally, while the variation of stimulation system (200) described above in connection with FIG. 2A uses a single reference electrode (208) (e.g., the stimulation device housing (220) in each of the four monopolar pathways, it should be appreciated that a stimulation system may make use of more than one electrode (e.g., as a cathode) in a given pathway that is not co-located with the other electrode(s) in that pathway. For example, in some variations, a stimulation system may include any or all of a monopolar stimulation pathway comprising an electrode on the distal end of a brain lead and the housing of another implanted device; a monopolar pathway comprising an electrode on the distal end of a brain lead and some other conductive element located somewhere other than on a brain lead as a reference electrode; and a bipolar pathway using an electrode on the distal end of a brain lead and another electrode on the same lead or a different lead, or the like.

A benefit of using the RSES method may be that the amplitude of the stimulation current delivered through a given pathway is more predictable and repeatable than can be achieved with a simultaneous method. The impedance that characterizes a given anode/cathode pathway may vary from one pathway to the next, depending on the characteristics of the tissue through which the current passes. In a simultaneous method, where the current is split among several pathways with the objective of delivering the same pulse of a burst through each stimulation pathway at the same time, the actual amplitude of the current delivered through each pathway may be slightly different, based on different impedances. For example, in the simultaneous method, if a ±8 mA pulse is split among four pathways with the intention of delivering identical ±2 mA pulses at the same time through each pathway, if there are variations in impedances in the different stimulation pathways, each pathway may receive a pulse that varies from the intended ±2 mA pulse (for example, a first stimulation pathway having a higher impedance might receive only a ±1.8 mA and a second stimulation pathway having a lower impedance might receive a ±2.2 mA, while only two of the pathways actually deliver the ±2.0 mA pulse). In these instances, one or more stimulation pathways receive less than the desired ±2 mA pulse, which may reduce the effectiveness of the stimulation provided by the stimulation system. When a RSES method is used to deliver stimulation, only one stimulation pathway is receiving a pulse or a segment of a pulse at a given time during the burst, and impedance mismatches in the different pathways used should have no effect on the amplitude of the current that actually gets delivered through the tissue. Thus, because the RSES methods apply each pulse or pulse segment to a single stimulation pathway, the supplied current need not be divided between multiple stimulation pathways, and thus may provide a more uniform and predictable stimulation pulse or pulse segment to each of the stimulation pathways. Delivering pulses or pulse segments to each stimulation pathway individually ensures that the desired amount of current and pulse-width is delivered through the electrodes independent of the impedance of the tissue.

Returning to FIGS. 3A and 3B, a variation of a stimulation system (300) is shown which comprises a plurality of bipolar stimulation pathways. As shown there, the stimulation system (300) may comprise four electrodes (301)-(304) which may be electrically connected to a stimulation device (320) via a lead (306). As shown there, the stimulation system (300) may be programmed such that electrodes (301) and (302) may form a first bipolar stimulation pathway, and electrodes (303) and (304) may form a second bipolar stimulation pathway. While electrodes (301) and (303) are shown in FIG. 3A as being anodes and electrodes (302) and (304) are shown as being cathodes, these pairings may be reversed. Additionally, any combination of the electrodes (301)-(304) may be used to form any number of stimulation pathways. It should also be appreciated that in one or more instances, one or more of the electrodes (301)-(304) may form a part of a monopolar stimulation pathway, such as described above.

FIG. 3B shows a time graph of one manner in which a RSES method may be used to deliver a 16 Hz burst (326) of pulses (324) over 0.5 seconds. As shown in FIG. 3B, the pulses (326) may be delivered sequentially to the first and second bipolar stimulation pathways. In some variations, the stimulation device (320) may select the first bipolar stimulation pathway (301)-(302) and deliver a first pulse of the burst thereto, then may select the second bipolar stimulation pathway (303)-(304) and deliver a second pulse of the burst thereto. This may be repeated one or more times until every pulse of the burst (326) has been delivered. For example, when the stimulation system (300) is used to deliver a 0.5 second burst (326) characterized by a frequency of 16 Hz, the stimulation device (320) may repeat the pattern three more times, such that four pulses are delivered through each of the first and second stimulation pathways over the 0.5 second burst. It should be appreciated that the stimulation system (300) may be programmed to deliver any suitable burst or bursts to the bipolar stimulation pathways.

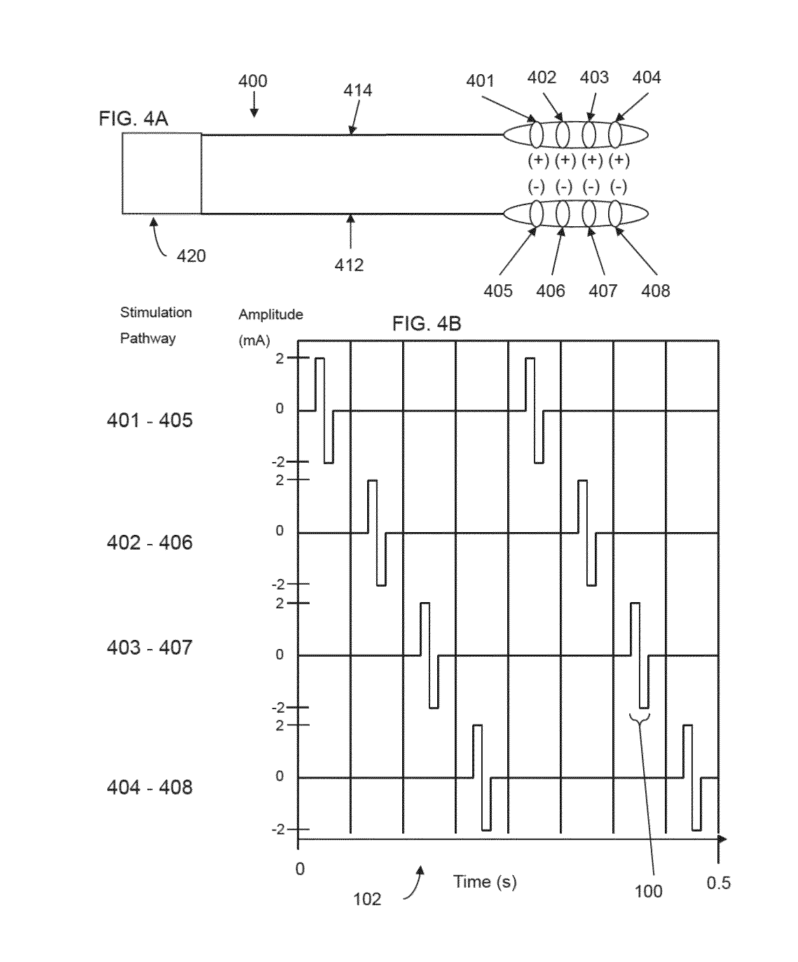

While each of the electrodes (301)-(304) of the stimulation system (300) are all located on the same lead, it should be appreciated that in some variations, electrodes of stimulation pathways may be contained on multiple leads. One such variation of a stimulation system will now be described with reference to FIGS. 4A and 4B. As shown in FIG. 4A, stimulation system (400) may comprise four electrodes (401)-(404) connected to a stimulation device (420) via a first lead (414) and four electrodes (405)-(408) connected to the stimulation device (420) via a second lead (412). As shown there, the stimulation system (400) may be programmed such that each electrode of the first lead (414) may be paired with an electrode of the second lead (412) to form four bipolar stimulation pathways. For example, the electrode pair (401)-(405) may act as a first stimulation pathway, the electrode pair (402)-(406) may act as a second stimulation pathway, the electrode pair (403)-(407) may act as a third stimulation pathway, and the electrode pair (404)-(408) may act as a fourth stimulation pathway. It should be appreciated, however, that the stimulation system (400) may be programmed to form different sets of stimulation pathways using the electrodes (401)-(408) as desired, and may be configured to deliver stimulation through these stimulation pathways using the RSES methods described here. Additionally, while each of electrodes (401)-(404) on first lead (414) are shown in FIG. 4A as being anodes and each of electrodes (405)-(408) of second lead (412) are shown as being cathodes, it should be appreciated that this designation may be switched for any or all of the stimulation pathways.

FIG. 4B shows a time graph of one manner in which a RSES method may be used to deliver the burst (102) of pulses (100) of FIGS. 1A and 1B to tissue via the stimulation system (400). The burst of pulses may be sequentially delivered to each of the four stimulation pathways described above, such that each pulse is delivered to one or the four stimulation pathways. Specifically, the stimulation device (420) may select a first stimulation pathway (401)-(405) and may deliver a first pulse thereto. The stimulation device (420) may then select a second stimulation pathway (402)-(406) and deliver a second pulse thereto. The stimulation device (420) may then select the third stimulation pathway (403)-(407) and deliver a third pulse thereto, and then select the fourth stimulation pathway (404)-(408) and deliver a fourth pulse thereto. This sequence may be repeated through the first to fourth stimulation pathways, until all of the pulses of the burst have been delivered to tissue. For example, when the stimulation system (400) is used to deliver a 16 Hz burst of pulses over half a second through the four pathways illustrated schematically in FIG. 4A, the stimulation device (420) may be configured to repeat the pattern a second time, such that two pulses are delivered to each of the first, second, third, and fourth stimulation pathways over the 0.5 second burst.

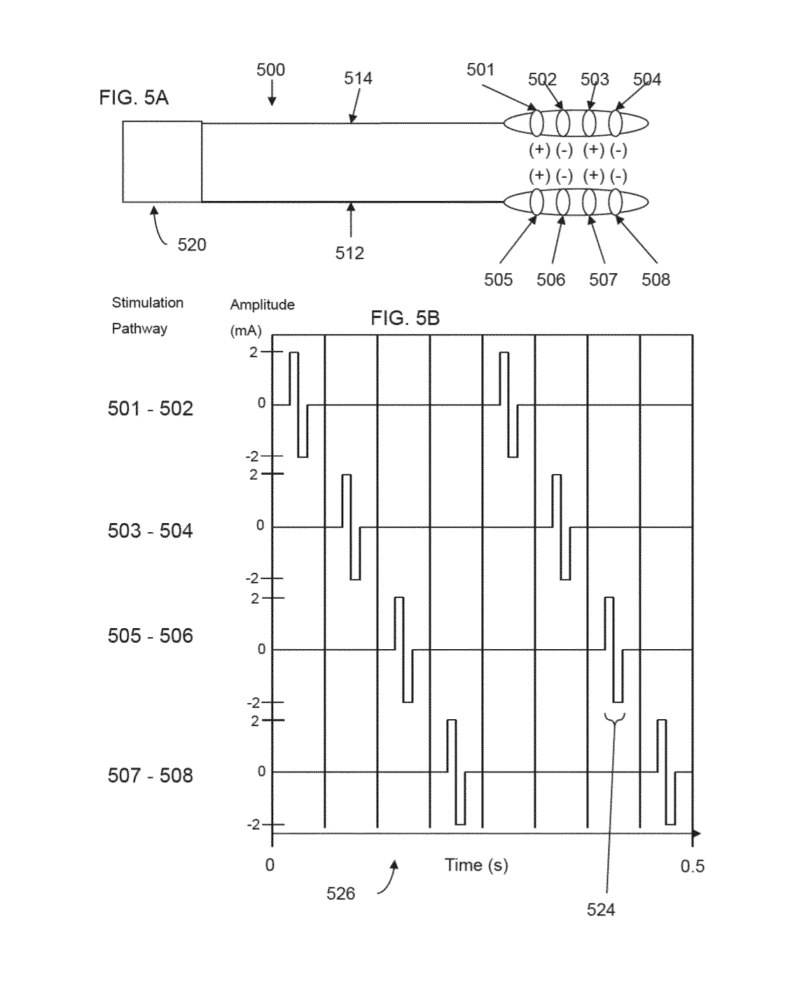

Another variation of a stimulation system is described with reference to FIGS. 5A and 5B. In this variation, a stimulation system may be programmed to designate a plurality of bipolar stimulation pathways that are configured from electrodes on two leads. As shown in FIG. 5A, the stimulation system (500) may comprise four electrodes (501)-(504) connected to a stimulation device (520) via a first lead (514) and four electrodes (505)-(508) connected to the stimulation device (520) via a second lead (512). As shown there, the electrodes (501)-(504) of first lead (514) may form two bipolar stimulation pathways, and the electrodes (505)-(508) of second lead (512) may form two bipolar stimulation pathways. The stimulation system (500) can be configured to deliver the pulses of a burst of stimulation through these pathways in any sequence, such as delivering first pulse through a bipolar pathway on the first lead followed by a second pulse through a bipolar pathway on the second lead, or a first pulse through a first bipolar pathway on the first lead followed by a second pulse through a second bipolar pathway on the first lead, followed by a third pulse through a first bipolar pathway on the second lead, and a fourth pulse through a second bipolar pathway on the second lead. For example, in one variation, the electrode pairs (501)-(502) and (503)-(504) of the first lead (514) may form first and second stimulation pathways, respectively, for delivery of a first pulse followed by a second pulse, and the electrode pairs (505)-(506) and (507)-(508) of the second lead (512) may form third and fourth stimulation pathways, respectively, for a third pulse followed by a fourth pulse.

FIG. 5B shows a time graph of one manner in which a RSES method may be used to deliver a 16 Hz burst (526) of pulses (524) over a 0.5 second interval to tissue via the stimulation system (500). The burst (526) of pulses (524) may be sequentially delivered to each of four stimulation pathways, such that each pulse (524) is delivered to a single stimulation pathway. For example, the stimulation device (520) may select a first stimulation pathway (501)-(502) and may deliver a first pulse thereto. The stimulation device (520) may then select a second stimulation pathway (503)-(504) and deliver a second pulse thereto. The stimulation device (520) may then select a third stimulation pathway (505)-(506) and deliver a third pulse thereto, and then select a fourth stimulation pathway (507)-(508) and deliver a fourth pulse thereto. This sequence may be repeated until all of the pulses of the burst have been delivered to tissue. For example, when stimulation system (500) is used to deliver the 16 Hz burst (526) of pulses (524) over a 0.5 second interval, the stimulation device (520) may repeat the pattern a second time, such that two pulses are delivered to each of the first, second, third, and fourth stimulation pathways over the 0.5 second burst.

When a stimulation system comprises multiple leads, and is programmed and configured to designate and deliver pulses or pulse segments to one or more bipolar stimulation pathways on each lead according to the RSES methods described here, the stimulation system may be configured to deliver pulses or pulse segments through the stimulation pathways in any suitable order. In some variations, a first round of a stimulation sequence may require pulses or pulse segments to be sequentially delivered to one or more stimulation pathways that are formed using the electrodes on the distal end of a first brain lead, and a subsequent round of the sequence may require pulses or pulse segments of a burst to be sequentially delivered to one or more stimulation pathways that are formed using the electrodes on the distal end of a second lead, and so on, for as many leads as may be appropriate. For example, in the RSES variation shown in FIG. 5B, pulses may be sequentially delivered to each of the stimulation pathways as may be formed using electrodes on the first lead (514), then may be sequentially delivered to each of the stimulation pathways as may be formed using electrodes of the second lead (512), such that each pulse is delivered to one stimulation pathway. In other variations, the method may comprise delivering pulses sequentially to each of the stimulation pathways as may be formed using electrodes on the second lead (512), then sequentially delivering pulses to the stimulation pathways formed using the electrodes on the first lead (514). In still other variations, the method may comprise alternating pulse delivery between bipolar stimulation pathways on different leads. For example, in some of these variations, a first pulse may be delivered to a first bipolar stimulation pathway between two electrodes physically located on a first lead, a second pulse may then be delivered to a first bipolar stimulation pathway formed between two electrodes physically located on a second lead, a third pulse may then be delivered to a second stimulation pathway formed between two electrodes on the first lead, and a fourth pulse may then be delivered to a second stimulation pathway formed between two electrodes on the second lead. The different stimulation pathways on a given lead may be formed using the same or different ones of the available electrodes that are disposed on the lead.

While the stimulation systems (400) and (500) described above with respect to FIGS. 4A and 5A are each shown as having two leads, it should be appreciated that the stimulation systems described here may comprise stimulation pathways formed using electrodes located on any number of leads (e.g., one, two, three, or four or more leads). Stimulation systems may comprise any combination of monopolar and bipolar stimulation pathways. In variations that include one or more bipolar stimulation pathways, each bipolar stimulation pathway may be entirely contained on one lead (such as the programmed stimulation pathways of the stimulation system (500) described above with respect to FIG. 5A) or may be divided among multiple leads (such as the programmed stimulation pathways of the stimulation system (400) described above with respect to FIG. 4A). It should also be appreciated that the stimulation devices may be programmed to select any two electrodes as a stimulation pathway. As such, in some variations a set of electrodes may define a first plurality of stimulation pathways during delivery of a first burst of pulses, and the same set of electrodes may define a different set of stimulation pathways during delivery of a second burst of pulse. For example, in the variation of the stimulation system (500) shown in FIG. 5A, the stimulation system (500) may be programmed such that the electrodes (501)-(508) may define a first set of stimulation pathways for one or more bursts of pulses (for example, the electrode pairs (501)-(502), (503)-(504), (505)-(506), and (507)-(508) may each define a stimulation pathway), and may define a second set of stimulation pathways for one or more bursts of pulses (for example, the electrode pairs (501)-(504), (502)-(504), (505)-(508), and (506)-(507) may each define a stimulation pathway).

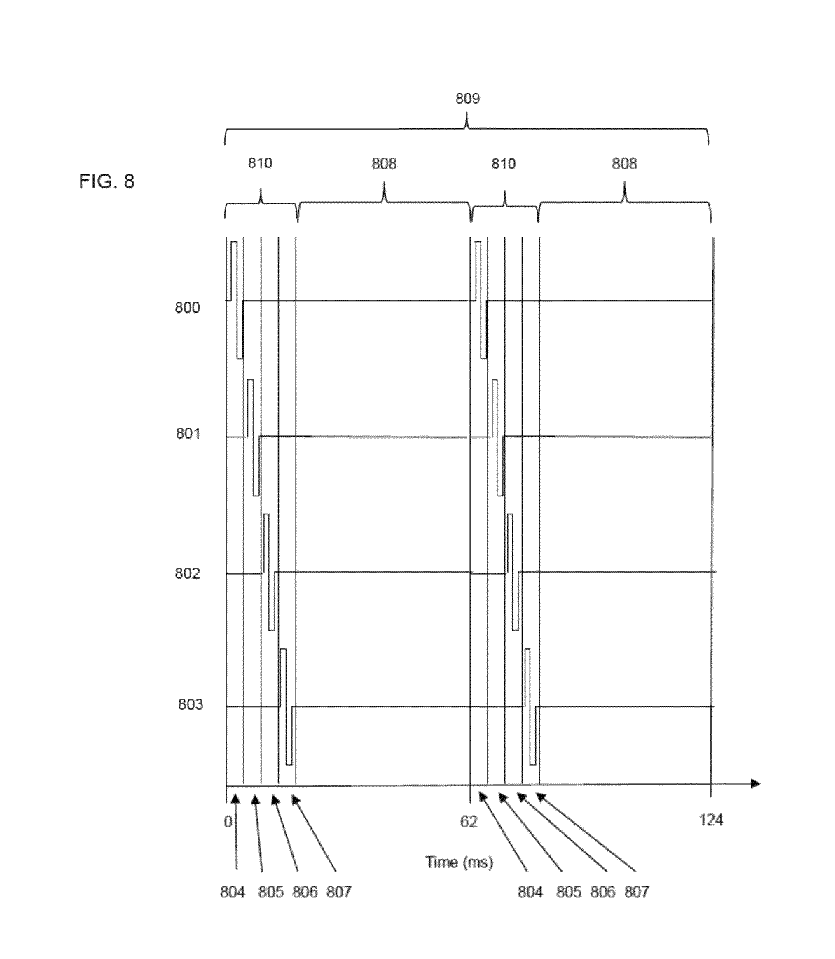

As mentioned above, in some instances, a stimulation system may be programmed such that the interval between successive pulses of a burst may vary over the course of delivery of the burst. For example, FIG. 8 shows a graph of one variation in which a RSES method may be configured to provide a burst of stimulation pulses to tissue to a plurality of stimulation pathways. A stimulation system (such as one or more of the stimulation systems described hereinthroughout) may be programmed and configured to selectively deliver stimulation through four stimulation pathways (labeled in FIG. 8 as a first stimulation pathway (800), a second stimulation pathway (801), a third stimulation pathway (802), and a fourth stimulation pathway (803)). Each of the stimulation pathways (800)-(803) may be a monopolar or bipolar stimulation pathway, as described in more detail above. In the RSES method shown in FIG. 8, the stimulation system may be configured to deliver a burst (809) of stimulation pulses to the stimulation pathways. The burst (809) of pulses may be divided into a plurality of pulse sequences (810) which are separated by inter-sequence intervals (808).

During each pulse sequence (810), a plurality of staggered pulses may be sequentially delivered to the stimulation pathways, such that each pulse is delivered to one stimulation pathway at each moment in time. In the variation of the pulse sequences (810) illustrated in FIG. 8, the stimulation device may be configured to deliver four pulses to the stimulation pathways during the pulse sequence (810), such that one pulse is delivered through each of the four stimulation pathways (800)-(803) and stimulation is delivered through only one of the four stimulation pathways (800)-(803) at a time. More specifically, the stimulation system may be programmed to deliver a first pulse (804) to tissue through the first stimulation pathway (800), then to deliver a second pulse (805) to tissue through the second stimulation pathway (801), then to deliver a third pulse (806) to the third stimulation pathway (802), and then to deliver a fourth pulse (807) to the fourth stimulation pathway (803). An inter-pulse interval of time may separate the delivery of each of the four pulses (804)-(807). In some instances, it may be desirable to begin delivery of the next pulse immediately after delivery of the previous pulse. In these instances, the inter-pulse interval between delivery of each of the four pulses (800)-(803) may be as close to zero as can be achieved by an output stage of a stimulation device.

Following the delivery of the pulse sequence (810), the stimulation device may be programmed to pause stimulation or otherwise wait during an inter-sequence interval (808), and then may be configured to re-deliver the pulse sequence (810) as described above. The delivery of the pulse sequence (810) through the four stimulation pathways (800)-(803), followed by the inter-sequence interval (808), may be repeated for the duration of the burst. The duration of the inter-sequence interval may set the frequency at which each pulse (810) sequence is delivered, and with it, the frequency that each of the four pulses (804)-(807) are delivered through each of the four stimulation pathways (801)-(803). For example, in an illustrative variation, the parameters of the burst may be programmed such that the pulse amplitude and duration of each of the four pulses (804)-(807) of the pulse sequence (810) may be ±2 mA and 320 μs, respectively. The parameters of burst (809) may be further configured such that delivery of each of the four pulses (804)-(807) may be separated by a 1 μs inter-pulse interval, and an inter-sequence interval (808) of approximately 61 ms may separate each sequence of the four pulses (804)-(807). In this variation, the total time for delivery of pulse sequence (810) (and with it, each of the four pulses (804)-(807)) and for the inter-sequence interval (808) is approximately 62.5 ms. Accordingly, when this pattern is repeated, it is repeated at a frequency of approximately 16 Hz. Additionally, each of the four pulses (804)-(807) is also delivered at a frequency of approximately 16 Hz, as each of the four pulses (804)-(807) is delivered once per 62.5 ms period.

The RSES method described above in relation to FIG. 8 may allow for more uniform and predictable current delivery than a simultaneous method, but without the need to reduce the frequency at which pulses are applied to each stimulation pathway. As noted above, in a simultaneous method, the current is split among several pathways with the objective of delivering the same burst through each pathway at the same time, and the actual amplitude of the current delivered through each pathway may be slightly different, based on different impedances. When the RSES method is used to deliver stimulation, only one pulse of one burst is ever being delivered at a given time during the burst, and impedance mismatches in the different pathways used should have no effect on the amplitude of the current that actually gets delivered through the tissue. Additionally, because the inter-sequence interval may be adjusted such that each staggered sequence of pulses is delivered at a desired frequency, the stimulation experienced by tissue may be substantially equivalent to the stimulation provided by the simultaneous method in terms of frequency of pulse delivery to each stimulation pathway, but without the potentially uneven current distribution between stimulation pathways. For example, in the simultaneous method, if a ±8 mA burst delivered at 16 Hz is split among four stimulation pathways with the intention of delivering four identical ±2 mA bursts at the same time through each pathway, each stimulation pathway may receive a 16 Hz burst that varies from the intended ±2 mA (depending on the impedance difference). Conversely, in the variation of the RSES method described immediately above, each of the four ±2 mA pulses (804)-(807) is delivered through respective stimulation pathways (the stimulation pathways (804)-(807)), at a rate of approximately 16 Hz.

While each pulse sequence (810) of the burst (809) provided by the RSES method described above with respect to FIG. 8 includes repeatedly delivering a pulse sequence (810) of four staggered pulses (804)-(807) to four stimulation pathways (800)-(803), it should be appreciated that the stimulation system may be configured to deliver a pulse sequence comprising any number of staggered pulses (e.g., two, three, four, five, or six or more pulses) to any number of stimulation pathways (e.g., two, three, four, five, or six or more pulses) in a sequence, where only one stimulation pathway receives a pulse at any given moment in time. For example, in some variations the stimulation system may be programmed to deliver burst to two stimulation pathways, where the burst includes delivery of a pulse sequence comprising two staggered pulses through the two stimulation pathways, such that one pulse is applied to each of the two stimulation pathways. In these variations, the stimulation system may be programmed to deliver a first pulse of the sequence to a first stimulation pathway. Following delivery of the first pulse (and in some variations, an inter-pulse interval), a second pulse of the pulse sequence is delivered to the second stimulation pathway. After the delivery of the pulse sequence of the two staggered pulses, the stimulation system may wait for an inter-sequence interval, and then may repeat the sequence. The two-pulse pulse sequence and the following inter-sequence interval may be repeated for the duration of the burst.

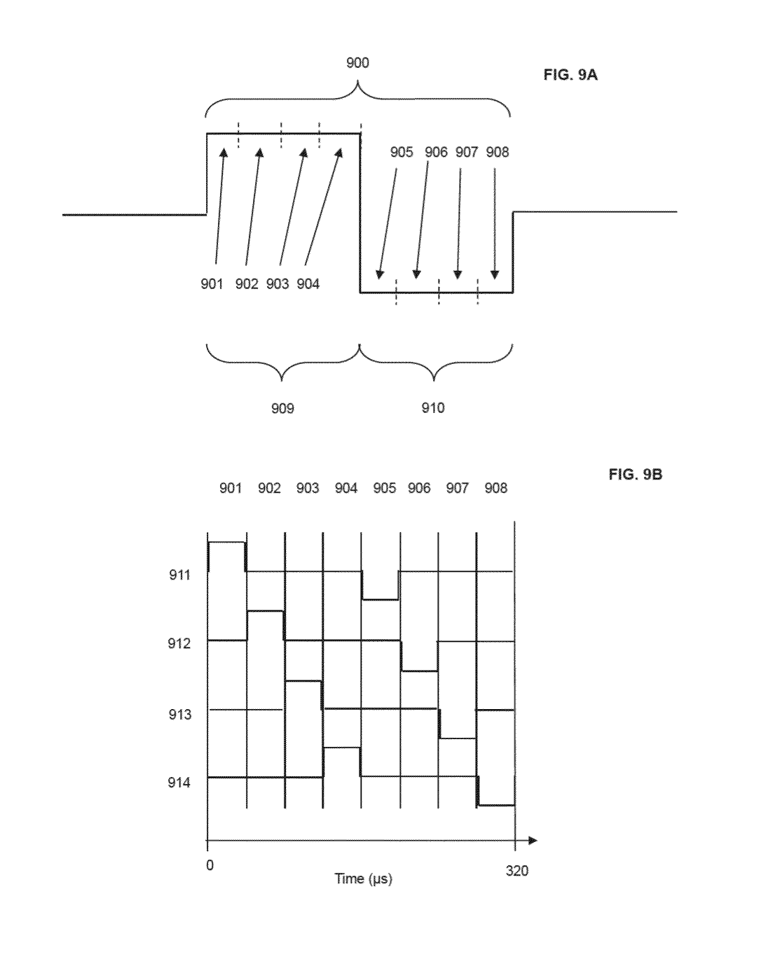

In some variations of the RSES methods described here, when a stimulation system is programmed and configured to deliver a stimulation pulses to tissue, the stimulation pulse may be time-division multiplexed among a plurality of stimulation pathways. In these variations, the multiplexed stimulation pulse may be divided into a plurality of “pulse segments”, and each pulse segment may be delivered through one of a plurality stimulation pathways. FIG. 9A shows an example of a pulse (900), which may be divided into a plurality of pulse segments. In a variation, the pulse (900) may have a pulse width of 320 μs, and may be divided into eight different 40 μs pulse segments (labeled in FIG. 9 as a first pulse segment (901), a second pulse segment (902), a third pulse segment (903), a fourth pulse segment (904), a fifth pulse segment (905), a sixth pulse segment (906), a seventh pulse segment (907), and an eight pulse segment (908)). Specifically, a positive phase (909) of the pulse (900) may be divided into four pulse segments (the pulse segments (901)-(904)), and a negative phase (910) of the pulse (900) may be divided into four pulse segments (the pulse segments (905)-(908)).

Each of the pulse segments (901)-(908) may be delivered to tissue through a single stimulation pathway selected from a plurality of stimulation pathways (e.g., two, three, four, or five or more stimulation pathways), such that stimulation is provided through one stimulation pathway at each moment in time. For example, FIG. 9B shows a method by which the eight pulse segments (901)-(908) of the pulse (900) may be delivered through four stimulation pathways (911)-(914) (which may be any combination of monopolar and bipolar stimulation pathways, as described in more detail above). As shown there, the first pulse segment (901) may be delivered to tissue through the first stimulation pathway (911), the second pulse segment (902) may then be delivered to tissue through the second stimulation pathway (912), the third pulse segment (903) may then be delivered to tissue through the third stimulation pathway (913), and the fourth pulse segment (904) may then be delivered to tissue through the fourth stimulation pathway (914). Following delivery of the fourth pulse segment (904), the fifth pulse segment (905) may be delivered to tissue through the first stimulation pathway (911), the sixth pulse segment (906) may then be delivered to tissue through the second stimulation pathway (912), the seventh pulse segment (907) may then be delivered to tissue through the third stimulation pathway (913), and the eighth pulse segment (908) may then be delivered to tissue through the fourth stimulation pathway (914). In this method, each stimulation pathway receives a pulse segment from the positive phase (909) of the pulse (900) and a pulse segment from the negative phase (910) of the pulse (900). A pulse segment from a positive phase (909) of the pulse (900) may be applied to an anode of a stimulation pathway, and a pulse segment from the negative phase (910) of the pulse (900) may be applied to a cathode of a stimulation pathway.

It should be appreciated that the pulse segments of a pulse may be delivered through any number of stimulation pathways (e.g., two, three, four, or five or more stimulation pathways) in any suitable order. Additionally, while the pulse (900) is shown in FIG. 9A as being divided into eight pulse segments (the pulse segments (901)-(908)), a pulse may be divided into any number of pulse segments (e.g., four, eight, sixteen, 32, 64, or more segments). Additionally, while each of the eight pulse segments (901)-(908) have a 40 μs duration, it should be appreciated that different pulse segments of a pulse may have different durations. For example, in a variation a pulse having a 320 μs pulse width may be divided into ten individual pulse segments, with six of the pulse segments having a 40 μs duration and four of the pulse segments having a 20 μs duration.

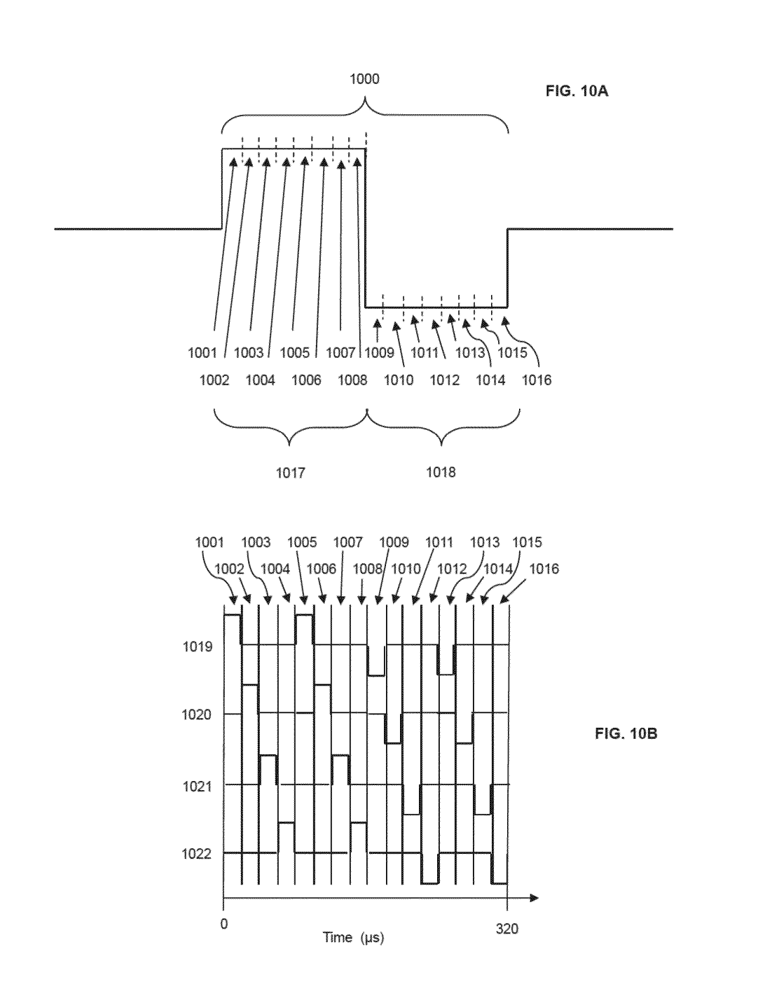

FIG. 10A shows one variation of a pulse (1000) which may be subdivided into sixteen pulse segments. In a variation, the pulse (1000) may have a pulse width of 320 μs, and may be divided into sixteen different 20 μs pulse segments (labeled in FIG. 10 as a first pulse segment (1001), a second pulse segment (1002), a third pulse segment (1003), a fourth pulse segment (1004), a fifth pulse segment (1005), a sixth pulse segment (1006), a seventh pulse segment (1007), an eighth pulse segment (1008), a ninth pulse segment (1009), a tenth pulse segment (1010), an eleventh pulse segment (1011), a twelfth pulse segment (1012), a thirteenth pulse segment (1013), a fourteenth pulse segment (1014), a fifteenth pulse segment (1015), and a sixteenth pulse segment (1016)). Specifically, a positive phase (1017) of the pulse (1000) may be divided into eight pulse segments (the pulse segments (1001)-(1008)), and a negative phase (1018) of the pulse (1000) may be divided into eight pulse segments (the pulse segments (1009)-(1016)). Each of the pulse segments (1001)-(1016) may be delivered to tissue through one of a plurality of stimulation pathways (e.g., two, three, four, or five or more stimulation pathways). For example, FIG. 10B shows a method by which the sixteen pulse segments (1001)-(1016) of the pulse (1000) may be delivered through four stimulation pathways (labeled in FIG. 10 as a first stimulation pathway (1019), a second stimulation pathway (1020), a third stimulation pathway (1021), and a fourth stimulation pathway (1022), which may be any combination of monopolar and bipolar stimulation pathways described in more detail above). As shown there, the first pulse segment (1001) may be delivered to tissue through the first stimulation pathway (1019), the second pulse segment (1002) may then be delivered to tissue through the second stimulation pathway (1020), the third pulse segment (1003) may then be delivered to tissue through the third stimulation pathway (1021), the fourth pulse segment (1004) may then be delivered to tissue through the fourth stimulation pathway (1022), the fifth pulse segment (1005) may delivered to tissue through the first stimulation pathway (1019), the sixth pulse segment (1006) may then be delivered to tissue through the second stimulation pathway (1020), the seventh pulse segment (1007) may then be delivered to tissue through the third stimulation pathway (1021), and the eighth pulse segment (1008) may then be delivered to tissue through the fourth stimulation pathway (1022). This pattern may then be repeated with the negative phase (1018) of the pulse (1000), with the ninth and thirteenth pulse segments ((1009) and (1013)) being delivered through the first stimulation pathway (1019), the tenth and fourteenth pulse segments ((1010) and (1014)) being delivered through the second stimulation pathway (1020), the eleventh and fifteenth pulse segments ((1011) and (1015)) being delivered through the third stimulation pathway (1021), and the twelfth and sixteenth pulse segments ((1012) and (1016)) being delivered through the fourth stimulation pathway (1022). In this method, each stimulation pathway receives two pulse segments from the positive phase (1017) of the pulse (1000) and two pulse segments from the negative phase (1018) of the pulse (1000).

When an individual pulse is time-division multiplexed, the delivery of pulse segments to a plurality of stimulation pathways may mimic the stimulation of simultaneous method in which a separate pulse is applied to each of the stimulation pathways simultaneously. For example, when the pulse (900) is time-division multiplexed such that eight pulse segments (901)-(908) are delivered to the four stimulation pathways (911)-(914), as shown in FIG. 9B, this time-division multiplexing may mimic the stimulation of a simultaneous method in which four separate pulses are simultaneously applied to four stimulation pathways such that each pathway receives an individual pulse. Because each pulse segment is delivered to only one stimulation pathway, no current division occurs, and thus impedance mismatches in the different pathways used should have no effect on the amplitude of the current that actually gets delivered through the tissue, as it would with the simultaneous method.

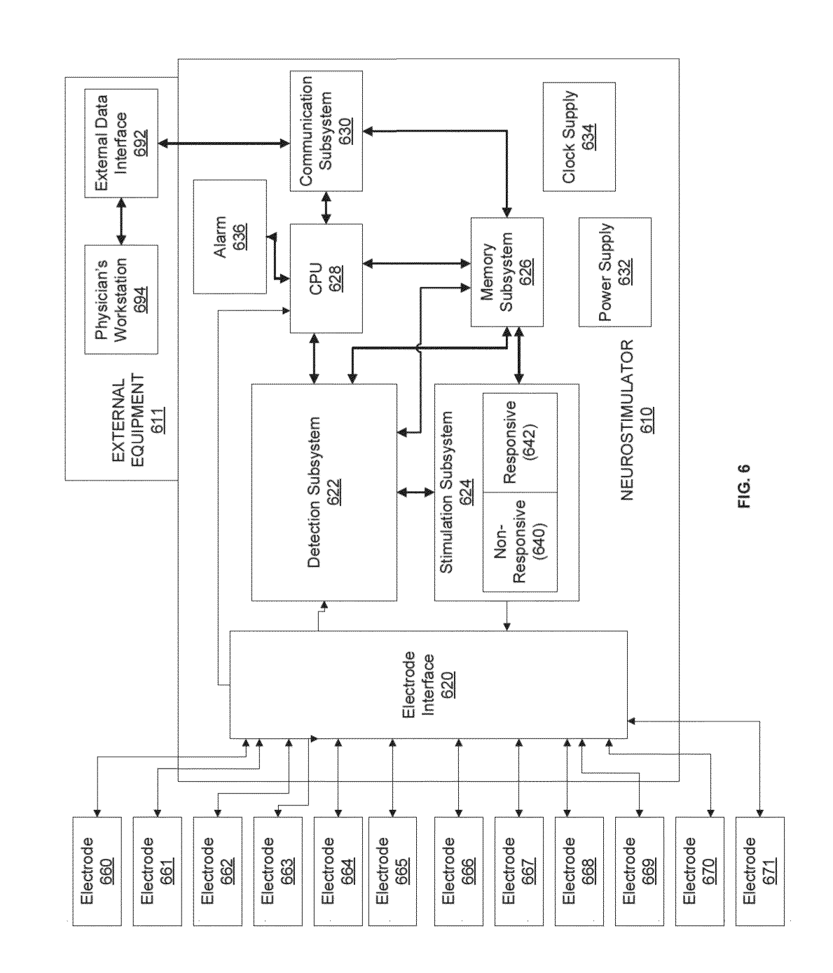

As mentioned above, the stimulation systems described here may include any suitable stimulation device. In some variations, the stimulation device may comprise one or more neurostimulators. Examples of neurostimulators that may be programmed and configured to deliver a burst of stimulation pulses via a RSES method may be found in U.S. Pat. No. 6,690,974, titled “Stimulation Signal Generator for an Implantable Device”, which is hereby incorporated by reference in its entirety. FIG. 6 depicts an overall block diagram of a variation of neurostimulator (610) which may be used to deliver one or more of the bursts described above. As shown there, neurostimulator (610) may comprise an electrode interface (620), a detection subsystem (622), a stimulation subsystem (624), a memory subsystem (626), a central processing unit (CPU) (628), a communication subsystem (630), a power supply (632), a clock supply (634), and an alarm (636). The neurostimulator (610) may be capable of being coupled to a plurality of electrodes (660)-(671) for sensing and/or stimulation. Each electrode may be configured to sense signals from neural tissue and/or may act as part of a stimulation pathway to deliver stimulation to tissue. It should be appreciated that a particular electrode may be used for both sensing and stimulation (e.g., an electrode may be used in a sensing configuration at one point in time, and may be used in a stimulation configuration at a different point in time). Although the neurostimulator (610) is described here with a sensing capability and a subsystem for detecting occurrences of features or events in the sensed signals, neither function is required of a neurostimulator configured to deliver sequential stimulation or in an RSES method.

The electrodes (660)-(671) may be connected to neurostimulator (610) via an electrode interface (620). Some or all of the electrodes may be electrically connected to neurostimulator electrode interface (620) via one or more leads (not shown). The electrode interface (620) may be coupled to the CPU (628), the detection subsystem (622) and the stimulation subsystem (624), and may be configured to select each electrode to act as a sensing electrode, act as an electrode in a stimulation pathway, or remain inactive (e.g., open or shorted). For example, a subset of electrodes of a neurostimulation system may be selected as sensing electrodes, and the electrode interface (620) may at least temporarily connect this subset of electrodes to the detection subsystem (622). This connection may allow detection subsystem (622) to receive one or more electrical signals (e.g., EEG signals, especially electrocorticographic signals (also sometimes referred to as ECoGs) obtained intracranially from the brain) from neural tissue via the sensing electrodes. Additionally or alternatively, a subset of electrodes of a neurostimulation system may be selected where each electrode is a node of a stimulation pathway, and the electrode interface (620) may at least temporarily connect this subset of electrodes to the stimulation subsystem (624), which may allow the stimulation subsystem (624) to selectively deliver stimulation one or more stimulation pathways. The CPU (628) may control which electrodes are selected as sensing electrodes and stimulation electrodes, and may direct the electrode interface (620) to switch an electrode between sensing, stimulation, or inactive configurations. Additionally, when the neurostimulator (610) is used to deliver each pulse of a burst of pulses sequentially to a plurality of stimulation pathways, as described in more detail above, the electrode interface (620) may select which stimulation pathway may receive each pulse. The electrode interface (620) may also provide one or more additional functions, including, but not limited to, signal amplification, electrode isolation, and charge-balancing functions, but it should be appreciated one or more of these functions may be also be achieved by one or more other subsystems of the neurostimulator. The selection of electrodes to be used in a given stimulation pathway, the segregation of pulses of a burst into different sequences for sequential delivery, and the timing of delivery of a burst or of pulses, pulse segments, or sequence of pulses within a burst may all be determined by programming the neurostimulator with parameters selectable from a menu or range of parameters.

As mentioned above, the neurostimulator (610) may include a detection subsystem (622) which may be configured to measure or otherwise monitor one or more physiological signals or discrete values sensed from a patient. The physiological information may include one or more electrophysiological signals (e.g., EEG signals (especially ECoGs)), temperature, blood pressure, and the like. Information regarding this information may be measured by the neurostimulator via one or more electrodes and/or sensors. For example, in the variation of neurostimulator (610) described above with respect to FIG. 6, the detection subsystem (622) typically comprises an EEG analyzer function which may analyze EEG signals sensed by one or more of the electrodes (660)-(671) (e.g., as selected by the CPU (628) and/or the electrode interface (620))

In some variations, the detection subsystem (622) may be configured to detect one or more predetermined criteria in the sensed physiological information. In variations where the neurostimulator comprises a responsive stimulation mode, the neurostimulator may be configured to deliver stimulation (e.g., one or more bursts) when one or more of the predetermined criteria have been detected. The predetermined criteria may comprise one or more patterns, threshold values, or combinations thereof. These criteria may be reflective of an occurring or imminent neurological event. For example, in variations where the detection subsystem (622) comprises an EEG analyzer function, the detection subsystem (622) may receive EEG signals from one or more of the electrodes (660)-(671). The detection subsystem (622) may process and analyze the received EEG signals to identify predefined features or events when these occur (e.g., in the time or frequency domain). The detected feature(s) or event(s) may correspond to or otherwise indicate a seizure, an onset of a seizure, a precursor to a seizure, a symptom of a movement disorder such as a tremor, an episode of depression, a migraine or cluster headache, or the like. EEG signal processing and analysis may comprise one or more signal processing techniques including, but not limited to, half wave counting, line length measurement, and area-under-the-signal calculations. The detection subsystem (622) may comprise one or more of the detection systems described in U.S. Pat. Nos. 6,016,449 to Fischell et al., for “System for Treatment of Neurological Disorders” issued Jan. 18, 2000 and 6,810,285 to Pless et al. for “Seizure Sensing and Detection Using an Implantable Device” issued Oct. 26, 2004, both of which are hereby incorporated by reference in its entirety.

As mentioned above, the neurostimulator (610) may comprise a stimulation subsystem (624) which may be configured to generate one or more electrical stimulation signals, which may be applied to neural tissue via one or more electrodes. Stimulation subsystem (624) may comprise a non-responsive portion (640) configured to generate one or more non-responsive stimulation signals, either continuously or periodically on a scheduled basis. Additionally or alternatively, stimulation subsystem (624) may comprise a responsive portion (642), which may generate one or more responsive stimulation signals when a predetermined criteria has been detected by the detection subsystem (622). It should be appreciated that the stimulation subsystem (624) and the detection subsystem (622) may be in communication (e.g., directly in communication, or in communication via the CPU (628)). In some instances, this communication may allow the detection subsystem (622) to blank one or more amplifiers or otherwise filter or process sensed signals during stimulation by the stimulation subsystem (624), as will be described in more detail below. Additionally or alternatively, this communication may allow the stimulation subsystem (624) to alter one or more parameters of a generated stimulation signal based one a signal received by the detection subsystem (622). In addition to the references cited previously, responsive neurostimulation for treating neurological disorders is described in, for example, U.S. Pat. No. 6,459,936 to Fischell et al. for “Methods for Responsively Treating Neurological Disorders” issued Oct. 1, 2002. Multimodal stimulation delivery and devices used to provide it are described in, for example, U.S. Pat. No. 6,466,822 to Pless for “Multimodal Neurostimulator and Process of Using It” issued Oct. 15, 2002 and U.S. Pat. No. 7,174,213 to Pless for “Electrical Stimulation Strategies to Reduce the Incidence of Seizures” issued Feb. 6, 2007. Each of these patents is incorporated by reference in its entirety.

The stimulation subsystem (624) may be programmed to generate any suitable electrical stimulation signal or combination of signals. The stimulation subsystem (624) may generate one or more bursts of pulsatile stimulation, and each pulse (or segment of a pulse, as described above) of a burst may be delivered to a single stimulation pathway using one or more of the RSES methods described above. The stimulation subsystem (624) may also be configured to generate one or more non-pulsatile (e.g., sinusoidal or quasi-sinusoidal waveforms), and/or DC signals. A stimulation output stage of a neurostimulator configurable to generate different varieties of stimulation is described, for example, in U.S. Pat. No. 6,690,974 to Archer et al. for “Stimulation Signal Generator for an Implantable Device,” issued Feb. 10, 2004. In some of these variations, these signals may also be delivered using one or more of the RSES methods described above, where the each pulse (or other meaningful division of a waveform, such as a phase in the case of a sinusoid) is delivered in sequence to a different one of more than one stimulation pathway, so that pulses (or phases or other units of the stimulation signal) are not being delivered simultaneously through the tissue through multiple pathways. For example, the stimulation signal may be delivered to a first stimulation pathway for a first time interval, to a second stimulation pathway for a second time interval following the end of the first time interval, and so on for each of the stimulation pathways, at which point the sequence of stimulation may repeated. For example, a method of stimulating tissue with a neurostimulator (610) may comprise delivering one or more stimulation signals using the methods described in U.S. Provisional Application No. 61/618,570, filed on Mar. 30, 2012 and titled “Low-Frequency Stimulation Systems and Methods”, which is hereby incorporated by reference in its entirety. As mentioned above, in variations that include a detection subsystem or function, one or more of the parameters of the stimulation provided by the stimulation subsystem (624) may be specified by one or more other subsystems of the neurostimulator (610). As mentioned above, one or more parameters of the stimulation signal may be determined at least partially by one or more parameters of a signal detected by the detection subsystem (622). U.S. Pat. No. 6,480,743 to Kirkpatrick et al. for “System and Method or Adaptive Brain Stimulation” issued Nov. 12, 2002 and U.S. Pat. No. 6,690,974 cited above, for example, describe methods of using features of a detected signal to determine parameters for stimulation. U.S. Pat. No. 6,480,743 is incorporated by reference in its entirety.

Additionally, some variations the stimulation subsystem (624) may further be configured to facilitate the administering to a patient one or more additional stimuli, other modulators of neurological activity (e.g., pharmaceuticals), and/or other types of therapy. For example, the stimulation subsystem (624) may be configured to provide a vibratory stimulus, an audio stimulus, and/or may be configured to dispense one or more drugs or therapeutic agents (e.g., via a drug dispenser (not shown)). These additional stimuli may be administered on a non-responsive basis and/or a responsive basis.

As mentioned above, the neurostimulator (610) may also comprise a memory subsystem (626) and a CPU (628), which in some instances may be a microcontroller. In a variation, the memory subsystem (626) may be connected to (i.e., in operable communication with) the detection subsystem (622) and configured to receive and store one or more EEG signals (such as signals sensed before, during, or after a form of stimulation, e.g., a burst of electrical stimulation, or an optical signal or electromagnetic or ultrasound therapy) or other data representative of a condition or state of the patient (e.g., a symptom of a disease, the disease itself, or a brain state (sleep or awake states)). The memory subsystem (626) also may be connected to the stimulation subsystem (624) (e.g., for storing and providing programmed stimulation parameters to the stimulation subsystem (624). The memory subsystem (626) further may be in operable communication with the CPU (628) (e.g., so that the CPU (628) may control the memory subsystem (626)). In variations where the neurostimulator (610) comprises a communication subsystem (630), the memory subsystem (626) may be connected to the communication subsystem (630), which may allow for data stored in the memory subsystem (626) (e.g., data relating to monitored EEG signals, stored EEG signals, stimulation parameters, and the like) to be uploaded to the external equipment (611). Additionally, information such as detection criteria and/or stimulation parameters may be downloaded to the memory subsystem (626) from the external equipment (611) via the communication subsystem (630).

Similarly, the CPU (628) may be connected to the detection subsystem (622), the stimulation subsystem (624), and/or the communication subsystem (630) for direct control over these subsystems. The CPU (628) may be connected to any suitable subsystem or functional portion of the neurostimulator (610) (e.g., an alarm (636)) and may be configured to control these subsystems and/or functional units. When two or more subsystems or functional units of the neurostimulator (610) are in operable communication, this connection may be analog or digital, and may be achieved using a single wire, a plurality of wires, wirelessly, or with any other suitable connection mechanism.

As noted above, the neurostimulator (610) may also comprise a communication subsystem (630). The communication subsystem (630) may be coupled to the memory subsystem (626) and/or the CPU (628), and may enable communication between the neurostimulator (610) and the external equipment (611). In the variation shown in FIG. 6, the external equipment (611) may comprise an external data interface (692) and a physician workstation (694). The external data interface (692) and the communication subsystem (630) may be configured to transmit data wirelessly. For example, in some variations the communication subsystem (630) may comprise a telemetry coil (which may or may not be positioned outside of the neurostimulator housing), which may allow for the transmission of signals from the communication subsystem (630) to the external data interface (692), or vice versa, via inductive coupling. Additionally or alternatively, the communication subsystem (630) may comprise an antenna which may provide an RF link between the communication subsystem (630) and the external data interface (692), or may comprise an audio transducer for an audio link.

The external data interface (692) may be coupled to the physician workstation (694) by a communication link, such as a data-transfer cable, a wireless connection, a phone line, an internet connection, or the like. The physician workstation (694) may be configured to upload and receive data from the neurostimulator (610) (e.g., data stored by the memory subsystem (626) corresponding to sensed signals, detected features or events (if the neurostimulator has a detection subsystem or detection functionality), device diagnostics (e.g., remaining power supply voltage, occurrences of device resets, etc.), or data measured in real-time from the sensing elements of the system (e.g., from electrodes configured to sense electrographic activity, such as field potential changes, or from sensors for other physiological information such as temperature, blood pressure, tissue oxygenation, etc.) or features or events detected in real time by the detection subsystem (622)). The physician workstation (694) may have some functionality to undertake various operations on data uploaded from the implanted neurostimulator (610) or other implanted components of a neurostimulation system (e.g., an implanted electrode-bearing deep brain lead or an implanted electrode-bearing cortical strip lead), such as to perform simulations on uploaded data to test whether various detection criteria will result in detecting desired features or events, such as neurological activity corresponding to electrographic onset of an epileptic seizure.

The physician workstation (694) may also be configured to download or transmit from the external equipment (611) to the neurostimulator (610) programming instructions (e.g., stimulation parameter values such as pulse amplitude and pulse width, frequency of pulses within a burst, interval between bursts, number of stimulation pathways to use in a given sequence of stimulation, detection criteria (if the neurostimulator has a detection subsystem or detection functionality), etc.), code and other information to the neurostimulator (610). The physician workstation (694) may further be configurable to command the neurostimulator (610) to perform specific actions (e.g., to record a portion of a monitored electrographic signal) or to change modes (e.g., from a detection-only mode to a responsive stimulation mode, or from a responsive stimulation mode to a scheduled stimulation mode, or from one of these to a combination of the other of these or from one combination to a different combination). Examples of external equipment suitable for use with the neurostimulation devices, systems, and methods describe here may be found in U.S. Pat. No. 6,810,285 to Pless et al. for “Seizure Sensing Device and Detection Using an Implantable Device” issued Oct. 26, 2004 (cited previously herein); U.S. Pat. No. 7,136,695 to Pless et al. for “Patient-Specific Template Development for Neurological Event Detection” issued Nov. 14, 2006; and U.S. Pat. No. 7,277,748 to Wingeier et al. for “Spatiotemporal Pattern Recognition for Neurological Event Detection and Prediction in an Implantable Device” issued Oct. 2, 2007. U.S. Pat. No. 7,819,812 for “Modulation and Analysis of Cerebral Perfusion in Epilepsy and Other Neurological Disorders” issued Oct. 26, 2010, describes external equipment including a programmer (a form of physician workstation) configurable to communicate with a plurality of implanted components (including programmable neurostimulators and leads) and other external equipment (e.g., a database) configurable to communicate with multiple programmers, which external equipment may be beneficially used with the systems, devices and methods described herein. Each of the patents cited above not previously incorporated by reference herein is hereby incorporated by reference in its entirety.

Additionally, the neurostimulator (610) may comprise a power supply (632) for supplying energy to one or more subsystems of the neurostimulator (610). In some variations, the power supply (632) may comprise a primary cell (non-rechargeable) battery. Additionally or alternatively, the power supply (632) may comprise a rechargeable battery. In some variations, the neurostimulator (610) may comprise one or more coils which may receive energy via magnetic induction from an external coil that may be placed in proximity to the coils of the neurostimulator. This energy received from the external coil (which may be positioned outside of the body) may be used to charge a rechargeable battery, or may directly power the neurostimulator (610) during the time the energy is being received by the neurostimulator. In one variation, the external coil may be used to establish a connection with the communication subsystem (630) or the neurostimulator (610) as described in more detail above. In some variations, one or more of the batteries may be associated with a DC-to-DC converter, which may be used to obtain a voltage larger than the voltage provided by the battery alone (e.g., a compliance voltage for a constant current stimulation output stage of neurostimulator (610)). U.S. Pat. No. 6,690,974 to Archer et al. for “Stimulation Signal Generator for an Implantable Device” issued Feb. 10, 2004 and previously cited and incorporated by reference herein, describes such a power supply. As previously described, the neurostimulator (610) may be configured to provide various forms of stimulation, modulation, and therapy. More specifically, with respect to electrical stimulation, the neurostimulator (610) may be configured to supply controlled current (also known as “current-controlled”) stimulation (by keeping a compliance voltage constant), controlled voltage (also known as “voltage-controlled” or “controlled-power”) stimulation, or controlled charge stimulation. See, e.g., Simpson et al., “An Experimental Study of Voltage, Current and Charge Controlled Stimulation Front-End Circuitry,” 2007, IEEE, the contents of which are hereby incorporated by reference herein.

Also shown in FIG. 6 is a clock supply (634). The clock supply (634) may supply any or all of the subsystems and other functional units of the neurostimulator (610) with any clock and timing signals that may be necessary for their operation. For example, the clock supply (634) may help synchronize different subsystems within the neurostimulator (610) or may provide time information for time- and date-stamping of neurological data sensed by the system and received, recorded, stored or otherwise obtained by the neurostimulator (610). In some variations, when the detection subsystem (622) of the neurostimulator (610) detects a neurological event or other predefined feature or condition in a sensed signal or other monitored value, the date and time of detection as well as other information relating to the occurrence may be stored in the memory subsystem (626) of the neurostimulator (610). The CPU (628) and/or the communication subsystem (630) may also provide data to the clock supply (634) to set the correct date and time of the clock supply (634).

While shown in FIG. 6 as having the alarm (636), the neurostimulator (610) need not. In variations that do comprise an alarm (636), the alarm (636) may be any component suitable for providing a perceivable stimulus to the patient (electrical stimulation delivered to neural tissue of the brain is generally not perceptible by a human patient). For example, in some variations the alarm (636) may be configured to deliver a tactile stimulus to the patient (e.g., vibration), an auditory stimulus (e.g., via a speaker), and/or another somatosensory signal (e.g., an electrical stimulation “tickle”) that may be perceived by the patient. The alarm (636) may be used to notify a patient of one or more conditions. In some variations, the alarm (636) may notify a patient that a particular neurological event has been detected. In other variations, the alarm (636) may notify a patient that the neurostimulator (610) is about to deliver stimulation to the patient. In still other variations, the alarm (636) may notify the patient of a condition of the neurostimulator (610), such as a low or insufficient power supply, a full or nearly-full memory subsystem (626), a device failure condition, or the like. It should be appreciated that the alarm (636) may be configured to deliver different stimuli to signify different information to a patient (e.g., a vibratory stimulus may be used to notify a patient of a low or insufficient battery supply, while an acoustic stimulus may be used to notify a patient that a neurological event has been detected). It should also be appreciated that in some variations, a stimulus provided by the alarm (636), such as an acoustic stimulus, may help terminate a neurological event (i.e., the alarm itself may constitute a therapy for a condition, such as aborting an epileptic seizure).

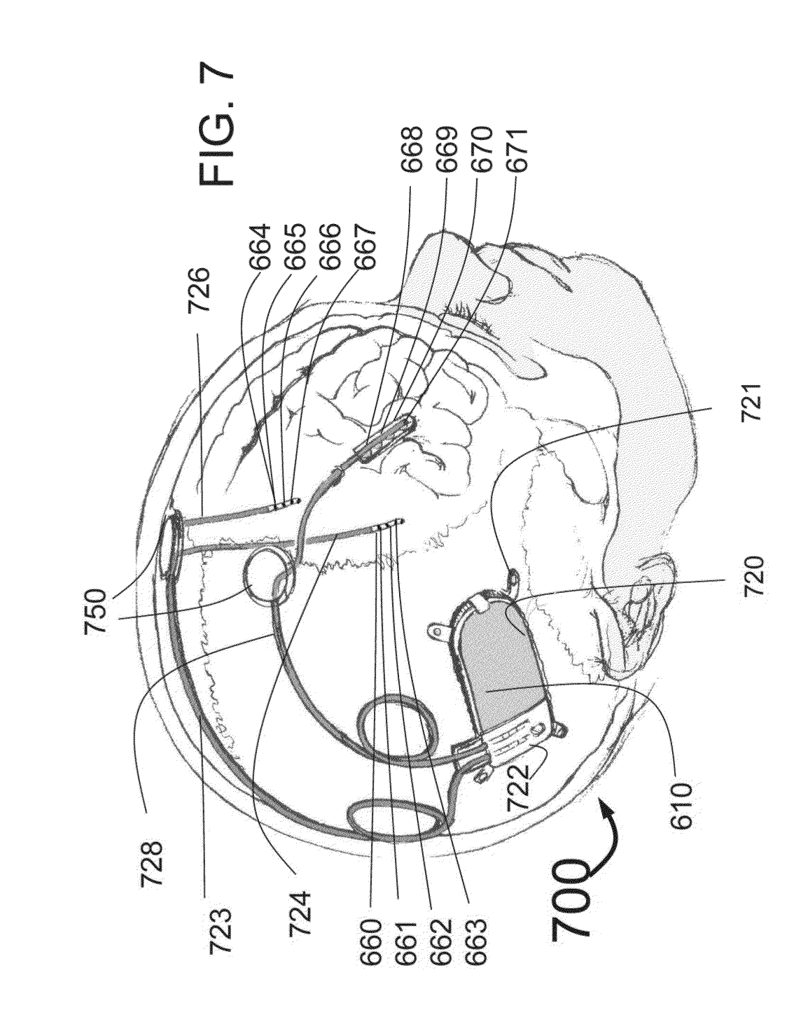

The neurostimulator (610) may be implanted, but need not be. In variations where the neurostimulator (610) is implantable, it may be implanted in any suitable location. In some variations, the neurostimulator (610) may be intracranially implanted. FIG. 7 shows one variation of an implantable neurostimulation system (700) in which the components of the neurostimulator (610) may be contained within an implantable housing (720). The housing (720) may be seated in an opening formed in the patient's cranium (for example, an opening formed by a craniotomy) and may additionally be anchored or otherwise attached to the cranium using one or more fasteners (721) (e.g., one or more bone screws, or the like). Additionally, in the variation shown in FIG. 7, the neurostimulation system (700) may comprise a first bifurcated lead (723) having a first bifurcation (724) and a second bifurcation (726), and second cortical strip lead (728). The first lead (723) and the second lead (728) may each be coupled to neurostimulator (610) at a lead connector or lead interface (722). The two bifurcations (724) and (726) of the first lead (723) and the second lead (728) each may access the brain through one or more burr holes (750) (two burr holes (750) are shown in FIG. 7) formed in the cranium, and may be used to position one or more electrodes (660)-(671) positioned on a distal portion of a lead relative to the brain.

While shown in FIG. 7 as comprising a bifurcated depth lead (723) (with first and second bifurcations (724) and (726)) and a cortical strip lead (728), the neurostimulation system (700) may comprise any suitable number of leads (e.g., one, two, three, or four or more leads). Additionally, each lead may comprise any suitable number of electrodes or other probes or transducers for sensing and/or stimulation or neuromodulation or other therapy. In the variation of neurostimulation system (700) shown in FIG. 7, the first lead (723) may comprise a furcated lead similar to the furcated lead described in U.S. Pat. No. 6,597,953 to Boling et al. for “Furcated Sensing and Stimulation Lead” issued Jul. 22, 2003. A depth lead (also referred to as a “deep brain lead”) typically has one or more electrodes positioned at a distal portion thereof which are designed to contact neural tissue in a region or structure of the brain (e.g., a region comprising or near a hippocampus of the brain). In FIG. 7, the first lead (723) is shown as a bifurcated depth lead with a uniform proximal lead portion and two furcated portions ((724) and (726)). More particularly, the first depth lead (723) of FIG. 7 is shown as provided with four cylindrical electrodes (i.e., the electrodes (660)-(663)) on the first bifurcation (724) and another four cylindrical electrodes (i.e., the electrodes (664)-(667)) on the second bifurcation (726). The portion of lead (723) proximal of the bifurcations (724) and (726) may extend out of one of burr holes (750) and is mechanically and electrically connected to the neurostimulator (610) via the lead connector (722) and conductors (not shown) extending through the lead from the electrodes (660)-(667), respectively. The cortical strip lead (728) may include four disc electrodes (668)-(671) on a proximal portion thereof. A cortical strip lead may be placed so as to position one or more electrodes on a surface of the brain (e.g., the cerebral cortex where it is believed that a focus of a partial epileptic seizure might exist when a patient has a seizure). Although in FIG. 7, a proximal portion of the cortical strip lead (728) is shown as extending out through one of the two burr holes (750), it will be appreciated that cortical strip leads often may be implanted in a patient without using a burr hole (750), and positioned so that one or more electrodes on a distal portion thereof are located under the dura mater and against or adjacent a surface of a patient's brain. Electrode-bearing leads for sensing electrographic activity and providing stimulation (especially electrical stimulation) to a patient's brain are described in U.S. Pat. No. 7,146,222 for “Reinforced Sensing and Stimulation Leads and Use in Detection Systems” issued Dec. 5, 2006. U.S. Pat. No. 6,944,501 to Pless for “Neurostimulator Involving Stimulation Strategies and Process for Using It,” issued Sep. 13, 2005 describes some of the considerations that may be involved in determine where to position electrodes for sensing electrographic information from the brain and delivering electrical stimulation to the brain and the selection of stimulation parameters based on the location of electrodes. Both of these patents are incorporated by reference herein in its entirety. U.S. Pat. No. 7,277,748 to Wingeier et al. for “Spatiotemporal Pattern Recognition for Neurological Event Detection and Prediction in an Implantable Device” issued Oct. 2, 2007 (cited and incorporated by reference previously) describes spatiotemporal considerations when electrodes are used to sense information from the brain. Each lead may be positioned so that the electrodes thereon are situated at any suitable location or locations in the brain. Examples of suitable electrode placement sites may include, but are not limited to the right and left hippocampus, right and left mesial temporal lobe, and the right and left anterior thalamus.

Although twelve electrodes (660)-(671) are shown in FIGS. 6 and 7, it should be recognized that the neurostimulation systems described here may comprise any suitable number of electrodes. For example, in some variations the neurostimulation system may comprise two, three, four, five, six, seven, eight, nine, ten, eleven, twelve, thirteen, fourteen, or fifteen or more electrodes. Additionally or alternatively, one or more portions of the neurostimulator housing (720) may act as an electrode, which may serve as a reference electrode for a monopolar stimulation pathway. For example, the housing (720) may comprise one or more conductive exterior portions, which may act as an electrode in use of the neurostimulator (610). Generally, each electrode may be selected (e.g., via the CPU (628) and/or the electrode interface (620)) and configured to act as either a sensing electrode (e.g., to sense one or more electrical signals the patient) or as an anode or cathode of a stimulation pathway (e.g., to deliver one on or more stimulation signals through an anode/cathode stimulation pathway to the brain). For example, in the variation of stimulation subsystem (700) shown in FIG. 7, the stimulation system (700) may be programmed and configured to use some or all of the electrodes (660)-(663) of the first bifurcation (724) and the electrodes (664)-(667) of the second bifurcation (726) to from a plurality of stimulation pathways (e.g., one or more monopolar stimulation pathways and/or one or more bipolar stimulation pathways), while the electrodes (668)-(671) of the cortical strip lead (728) may be configured to act as sensing electrodes. It should be appreciated, however, that an electrode may act as a sensing electrode in one instance, and form part of a stimulation pathway in another.

While particular embodiments and applications of the present invention have been illustrated and described herein, it is to be understood that the invention is not limited to the precise construction and components disclosed herein and that various modifications, changes, and variations may be made in the arrangement, operation, and details of the methods and apparatuses of the present invention without departing from the spirit and scope of the invention as it is defined in the appended claims.

1: A neurostimulator assembly for providing rapid sequential electrode stimulation to a patient comprising:
a) a plurality of electrodes; and
b) a stimulation device comprising a stimulation subsystem,
wherein the stimulation subsystem is programmed to generate a burst of electrical stimulation comprising a plurality of pulses and sequentially introduce the plurality of pulses to a plurality of stimulation pathways, wherein each stimulation pathway is defined by an anode and a cathode, and
wherein the stimulation device is programmed to time the introduction of the plurality of pulses such that each pulse is introduced through only one of the plurality of stimulation pathways.

2: The system of claim 1 wherein the stimulation device is programmable to select the plurality of stimulation pathways from a group of possible stimulation pathways.

3: The system of claim 2 wherein the burst is characterized by a set of burst parameters including burst duration, pulse parameters, and frequency of pulse delivery, and wherein the stimulation device is further programmable to predefine the set of burst parameters.

4: The system of claim 1 wherein the stimulation device is further configured to divide the burst into a plurality of pulse sequences, wherein each pulse sequence comprises introduction of a subset of the plurality of pulses, and wherein the stimulation device is configured to time delivery of the plurality of pulse sequences such that immediately sequential pulse sequences are separated by an inter-sequence interval.

5: The system of claim 4 wherein delivery of each pulse sequence comprises sequentially delivering a pulse to each of the plurality of stimulation pathways.

6: A method of providing rapid sequential electrode stimulation to a patient comprising:
generating a burst of electrical stimulation comprising a plurality of pulses; and
sequentially introducing the plurality of pulses to a plurality of stimulation pathways, wherein each stimulation pathway is defined by an anode and a cathode;
wherein the plurality of pulses are introduced such that each pulse is introduced through only one of the plurality of stimulation pathways.

7: The method of claim 6 wherein introducing the plurality of pulses comprises delivering a plurality of pulse sequences, wherein delivery of each pulse sequence comprises introducing of a subset of the plurality of pulses to the plurality of stimulation pathways, and wherein delivery of immediately sequential pulse sequences is separated by an inter-sequence interval.

8: The method of claim 7 wherein delivery of each pulse sequence comprises sequentially delivering a pulse to each of the plurality of stimulation pathways.

9: A neurostimulator assembly for providing rapid sequential electrode stimulation to a patient comprising:
a) a plurality of electrodes; and
b) a stimulation device comprising a stimulation subsystem,
wherein the stimulation subsystem is configured to generate a burst of electrical stimulation comprising a plurality of pulses and to sequentially introduce the plurality of pulses to a plurality of stimulation pathways, wherein each stimulation pathway is defined by an anode and a cathode,
wherein the stimulation subsystem is further configured to divide each pulse into a plurality of pulse segments, and
wherein the stimulation device is configured to time the introduction of the plurality of pulses such that each pulse segment is introduced through only one of the plurality of stimulation pathways.

10: A method of providing rapid sequential electrode stimulation to a patient comprising:
generating a burst of electrical stimulation comprising a plurality of pulses; and
sequentially introducing the plurality of pulses to a plurality of stimulation pathways, wherein each stimulation pathway is defined by an anode and a cathode;
wherein the plurality of pulses are introduced such that each pulse is divided into a plurality of pulse segments, each segment is introduced through only one of the plurality of stimulation pathways.
